(19) United States
(12) Patent Application Publication
(10) Pub. No.: US 2017/0079989 A1
(43) Pub. Date: Mar. 23, 2017

(54) METHOD FOR TREATMENT OF OSTEOARTHRITIS AND OTHER JOINT RELATED INJURIES AND CONDITIONS

(71) Applicant: Thomas Q. Dinh, Minnetonka, MN (US)

(72) Inventor: Thomas Q. Dinh, Minnetonka, MN (US)

(21) Appl. No.: 15/253,856

(22) Filed: Aug. 31, 2016

Related U.S. Application Data
(62) Division of application No. 12/907,934, filed on Oct. 19, 2010, now abandoned.

(51) Int. Cl. A61K 31/635; A61K 45/06; A61K 47/26; A61K 47/36; A61K 9/06; A61K 31/12; A61K 9/51; A61K 9/00; A61K 47/20

(52) U.S. Cl. CPC A61K 31/635; A61K 9/0019; A61K 45/06; A61K 47/26; A61K 47/20; A61K 9/06; A61K 31/12; A61K 9/5123; A61K 47/36

(57) ABSTRACT

The methods using associated formulations for intra-articular local delivery to treat an afflicted joint, for example, to treat osteoarthritis. Some methods deliver anti-inflammatory agents and angiogenesis inhibitors, and can include sensitizing agents for one or both. Formulations can include hyaluronic acid, a therapeutic solvent, and drug micro or nanoparticles, which may be formed of drug alone or in combination with an excipient or polymeric carrier. The carrier can include hydrophobic sugar nano-particles, which can resist degradation and provide extended cushioning. The excipient or polymer can be used to manipulate release rates and to increase drug retention to the affected region.

### RELATED APPLICATIONS

The present application claims priority to application Ser. No. 12/907,934 as a Divisional application electing the non-elected claims pursuant to a Restriction Requirement. application Ser. No. 12/907,934 claims priority to U.S. Provisional patent application No. 61/252,765, filed Oct. 19, 2009, titled Pharmaceutical Compositions and Methods for Treatment of Osteoarthritis.

The present application incorporates all contents of application Ser. No. 12/907,934, filed on Oct. 19, 2010, by reference.

### FIELD OF THE INVENTION

The present invention is related generally to pharmaceutical compositions and methods for treating joints. More specifically, the present invention includes compositions and methods for intra-articular delivery to treat injured joints, including those afflicted by osteoarthritis.

### BACKGROUND

Osteoarthritis is a group of joint diseases that represent a major burden for patients as well as societies as a whole. The progressive degenerative damage of articular cartilage observed in osteoarthritis is based on a complex etiology that is still insufficiently clear. Biochemical alterations, genetic and environmental factors converge to the manifestation of osteoarthritis (footnoted reference cites to this and other background information are provided in the related provisional patent application). Accumulating evidence indicates that beyond cartilage surrounding tissues such as the subchondral bone and the synovium are involved in the initiation and progression of osteoarthritis. In the recent years it has become increasingly clear that mechanisms of inflammation and angiogenesis contribute to osteoarthritis and might play an important causal role in the disease progression. Articular cartilage is essentially avascular and, therefore, requires adjacent highly vascularized structures for metabolic support that is mediated by the subchondral bone and the synovium.

This is important for maintaining structural integrity of the tissue and to permit high mechanical loading. Interestingly, articular cartilage explants are resistant to vascular invasion in vitro.

Thus, healthy cartilage exhibits the ability to prevent blood vessel ingrowth. Although the underlying mechanisms are not well known the expression of antiangiogenic mediators likely contributes to this phenomenon. The concept of involvement of angiogenesis in osteoarthritis has been developed several years ago, but did not receive much attention, while the role of angiogenesis in tumor growth became widely accepted.

However, in recent years the importance of angiogenesis in osteoarthritis has been clearly recognized. In osteoarthritis the invasion of blood vessels from the subchondral bone is apparent even in early stages of the disease and subsequently leads to the loss of tidemark integrity. This is accompanied by new bone formation at the osteochondral junction, a process that is believed to originate as a tissue response due to the altered biomechanical environment in the diseased joint. In addition, sensory nerve formation is observed in osteoarthritis explaining the development of pain, based on the fact that angiogenesis and innervation are regulated by similar mechanisms. Due to the lack of blood vessels, articular cartilage is characterized by a low oxygen tension. Since hypoxia is an important positive regulator of angiogenesis, healthy cartilage is considered to maintain a mechanism of hypoxia resistance although the underlying molecular events are unclear at present. Nevertheless, in osteoarthritis oxygen tension appears to be even lower compared to healthy cartilage, which likely contributes to the angiogenic processes. Recently, the role of inflammation in the degradative processes in osteoarthritis is increasingly recognized. Histologically, chronic synovitis is observed in patients undergoing joint replacements. Macrophage infiltration, endothelial proliferation and increased angiogenesis in the synovium apparently contribute to the disease progression.

Inflammation and angiogenesis are closely linked in a synergistic manner and, at least in part, are based on similar mechanisms. In osteoarthritis, inflammation induced angiogenesis has recently evolved as an important contributor to disease progression in a more exacerbated manner than previously thought.

Inflammation is a fundamental biological process consisting of a dynamic complex of cytological and chemical reactions that occur in the affected blood vessels and adjacent tissues in response to an injury or abnormal stimulation caused by a physical, chemical, or biological agent. The process of inflammation includes: 1) local tissue reactions and resulting morphologic changes; 2) destruction or removal of injurious material; and 3) responses that lead to repair and healing. The so-called “cardinal signs” of inflammation are redness, heat (or warmth), swelling, pain and inhibited or lost function. All of these signs may be observed in certain instances, but no one of them is necessarily always present. A disease that involves inflammation is herein referred to as an “inflammatory disease”.

Arthritis is an inflammatory disease characterized by inflammation of a joint, which term includes synovial tissue and membranes. There are many forms of arthritis, including without limitation, osteoarthritis (hypertrophic or degenerative arthritis), rheumatoid arthritis, arthritis due to infection (tuberculosis, Lyme disease, rheumatic fever, etc.), suppurative arthritis, juvenile arthritis, and gouty arthritis. Elevated tissue levels of IL-I, IL-8, and TNF are found in arthritis and in other inflammatory conditions.

In osteoarthritis, the cartilage that covers the ends of the bones that form the joint is slowly degraded by the action of various enzymes, particularly the matrix metalloproteinases (MMPs) which are secreted into the synovial fluid of the joint by the synovial lining cells in response to stimulation by various proinflammatory cytokines, particularly Interleukins (IL-1, IL-6, IL-8, IL-18) and TNF. The destruction of cartilage by the MMPs perpetuates the inflammatory reaction and leads to the joint pain associated with osteoarthritis (Bonnet and Walsh, Reumatology 2005, 44 (1))

Angiogenesis—the generation of new blood vessels by sprouting from preexisting blood vessels depends on a complex network of activators and inhibitors that are regulated in a timely and sequentially order to mediate blood vessel formation. Principally, this leads to nutrient delivery, maintenance of oxygen homeostasis, supports the removal of waste products, allows for tissue regeneration and provides biological mediators. In the healthy adult angiogenic processes mainly represent a repair system (e.g. fracture healing), thus the ingrowth of blood vessels into articular cartilage can be viewed as a failed repair process.

Inflammatory mediators can either directly or indirectly stimulate angiogenesis. Inflammatory cells that produce these factors include the macrophages and mast cells that are present in abundance in chronically inflamed osteoarthritic synovium. There are some general mechanisms by which macrophages can induce angiogenesis. New vessel growth can be stimulated directly by factors secreted from macrophages [Sunderkotter et al., Pharmacol Ther, 1991]. Macrophages can be found in most sites where abnormal angiogenesis is occurring, for example in synovitis and in tumors. Many of the inflammatory mediators produced by these macrophages induce angiogenesis in vivo. Macrophages can also secrete factors that stimulate other cells, such as endothelial cells and fibroblasts, to produce angiogenic factors such as VEGF [Lingren, Arch Pathol Lab Med, 2001; Cohen et al. J. of Biochem 1996, Ben-Av et al., FEBS Lett 1995].

Although not as well defined, neutrophils and lymphocytes have also been implicated in the induction of angiogenesis. Angiogenic factors such as basic fibroblast growth factor (bFGF) and VEGF may be produced by lymphocytes, and neutrophils may be involved in the early induction of angiogenesis [Lingren, Arch Pathol Lab Med, 2001, Blatnick et al., Proc Natl Acas Sci USA 1994].

As well as inflammatory cells, inflammatory conditions can also stimulate angiogenesis. Tissue hypoxia often occurs in inflamed tissue and is a potent stimulator of angiogenesis. VEGF gene expression is up-regulated during hypoxia and it is thought that this stimulation of angiogenic factors is an attempt to relieve the low oxygen content of the tissue [Jackson et al., J Reumatol 1997].

Angiogenesis is observed in the synovium of osteoarthritic joints, closely associated with chronic synovitis [Walsh and Haywood, Curr Opin lnvestig Drugs 2001; Giatromanolaki et al. J Pathol 2001]. The normal synovium is highly vascular in order to supply the normally avascular cartilage with nutrients and oxygen. In OA, increased endothelial cell proliferation is associated with new vessel formation. Concurrent vascular regression results in little overall change in vascular density. Instead, there is a redistribution of vessels within the synovium and a change towards a more immature phenotype [Steven C R et al., Arthritis Rheum 1991]. Increased vascular turnover in the osteoarthritic synovium reflects a change in the balance between angiogenic and anti-angiogenic factors. The extent of endothelial cell proliferation increases with increasing vascular density, increased macrophage infiltration and increased VEGF expression within the synovium, indicating that synovial neovascularization may be largely driven by synovitis [Haywood L et al., Arthritis Rheum 2003]. Up-regulation of hypoxia inducible factor-Le in the osteoarthritic synovium is also associated with increased microvascular density and expression of angiogenic factors, indicating that hypoxia may play an additional mediating role [Giatromanolaki A et al., Arthritis Res Ther 2003].

Angiogenesis may be most important in potentiating or perpetuating inflammation rather than in initiating it. Adhesion molecules such as E-selectin are highly expressed by new vessels, facilitating inflammatory cell infiltration [Fox S B et al. J Pathol 1995]. The inflammatory response can also be maintained by new vessels transporting inflammatory cells, nutrients and oxygen to the site of inflammation. It is also thought that deficient neural and peptide regulatory factors in the neovasculature may impair the vascular regulation of inflammation [Walsh D A et al. Histochem J 1996]. Angiogenesis may indirectly promote itself by increasing inflammatory cell infiltration, thereby increasing the availability of angiogenic factors produced by these cells. During early synovitis, angiogenesis may contribute to the transition from acute to chronic inflammation [Walsh D A, Rheumatology 1999].

Angiogenesis occurs at the osteochondral junction as well as within the osteoarthritic synovium. Vascularization of the articular cartilage and osteophytes is characteristic of the pathology of OA [Pufe T et al. Arthritis Rheum 2001]. The normal articular cartilage is avascular in the adult. A deep layer of calcified cartilage lies between the tidemark and the osteochondral junction.

Blood vessels may penetrate the calcified cartilage within fibrovascular channels originating from the subchondral bone [Bromley M. et al. Ann Rheum Dis 1985)]. With increasing severity of OA, these vascular channels breach the tidemark, and blood vessels may be found more superficially in the non-calcified articular cartilage. Blood vessels within the deep layers of the osteoarthritic articular cartilage are derived from the vasculature that is normally present in subchondral bone.

Hyaluronic gels are currently used to treat pain in various cases of osteoarthritis (OA) of the knee. Three products, SYNVISC®, Hylan GF-20 (Genzyme Corporation. Cambridge, Mass.), HYALGAN®. Sodium Hyaluronate (Sanofi-Aventis U.S. LLC. New York. N.Y.), and SUPARTZ®. Joint Fluid Therapy (Smith & Nephew pie, London, England) have all received FDA approval for treatment of OA in patients who do not respond to NSAIDs. Of these three, SYNVISC® HylanGF-20 is the most widely used. Hyaluronic gels used to treat pain associated with OA of the knee use almost pure solutions of hyaluronic acid (HA), a biological polymer that is native to all mammalian connective tissue. For example, SYNVISC®. Hylan GF-20 includes 0.8% HA. Despite their success for treatment of OA of the knee, the treatment can only provide short-term relief and does not address the biological origin of the disease in OA regarding to angiogenesis and inflammation. HA is basically acted as a mechanical cushion to alleviate pain in OA treatment. As a result, the effect did not last long.

Despite the availability of a wide range of medications and treatment modalities for arthritis and inflammatory diseases in general, as described above, none has proved to be entirely satisfactory for osteoarthritis. In particular, there remains a need for innovative treatments that target the underlying cause of osteoarthritis.

### SUMMARY OF THE INVENTION

According to its major aspects and broadly stated, some embodiments of the present invention provide methods and compositions for intra-articular injection to treat abnormal angiogenesis, inflammation, pain and loss of function associated with osteoarthritis, by using certain materials and pharmaceutical active ingredients that are capable of providing means to minimize pain and inhibit angiogenic growth factors and cytokines that are responsible for forming abnormal angiogenesis and inflammation in joints.

One embodiment of the present invention includes a method for treating osteoarthritis, which includes directly administering to the affected joint, preferably by intra-articular injection (direct injection into the closed cavity of the joint), a therapeutically effective amount of at least one angiogenesis inhibitor, alone or in combination with at least one anti-inflammatory agent.

Angiogenesis and inflammation are closely integrated processes in osteoarthritis (OA) and may affect disease progression and pain. Inflammation can stimulate angiogenesis, and angiogenesis can facilitate inflammation. Angiogenesis can also promote chondrocyte hypertrophy and endochondral ossification, contributing to radiographic changes in the joint. Inflammation sensitizes nerves, leading to increased pain. Innervation can also accompany vascularization of the articular cartilage, where compressive forces and hypoxia may stimulate these new nerves, causing pain even after inflammation has subsided. Inhibition of inflammation and angiogenesis may provide effective therapeutics for the treatment of OA by improving symptoms and retarding joint damage.

Some methods of the present invention treat the underlying cellular processes that lead directly to pain and tissue destruction associated with osteoarthritis. In one embodiment, the invention includes administering to the synovial tissue an effective amount of pharmaceutical ingredient; i.e., an amount that is sufficient to reduce any or all of the symptoms of osteoarthritis without producing any of the undesirable side effects resulting from an overdose. Some embodiments of the present invention provide means for interfering with cell signaling by cytokines such as TNF-a, IL-1, IL-18, IL-4, and IL-8 and angiogenic growth factors such as VEGF (vascular endothelial growth factor) and bFGF (basic fibroblast growth factor), thereby leading to lower MMP levels and correspondingly lower cartilage destruction and resultant pain.

Inhibitors of angiogenesis in OA believed useful in some embodiments of the present invention include, but are not limited to, mTor inhibitors such as rapamycin, rapamycin analogs (Temsirolimus, Biolimus, Everolimus, Zotarolimus or ABT-578), curcumin, endostatin, angiostatin, canstatin, interferon-a and platelet factor 4, neovastat, 2-methoxy estradiol, Imatinib (Glivec), Trastuzumab (Herceptin) and Celebrex.

One embodiment method of administering angiogenesis inhibitor is to directly inject a pharmaceutically acceptable composition containing at least one angiogenesis inhibitor into the closed cavity of an arthritic joint. The angiogenesis inhibitor may be administered alone or in combination with other medicaments, preferably at least one anti-inflammatory agent commonly used to treat osteoarthritis. Osteoarthritis treatment agents include, but are not limited to pharmaceutically acceptable steroidal and non-steroidal anti-inflammatory agents.

In another embodiment, the angiogenesis inhibitor may be administered alone or in combination with other medicaments, preferably at least one NFKB inhibitor. NFKB inhibitors include curcumin, sulfasalazine, sulindac, indomethacin, diclofenal, etodolac, meclofenate, mefenamic acid, nambunetone, piroxicam, phenylbutazone, meloxicam, dexamethasone, betamethasone dipropionate, diflorsasone diacetate, clobetasol propionate, halobetasol propionate, amcinomide, beclomethasone dipropionate, fluocinomide, betamethasone valerate, triamcinolone acetonide, penicillamine, hydroxychloroquine, sulfasalazine, azathioprine, minocycline, cyclophosphamide, methotrexate, cyclosporine, leflunomide, etanercept, infliximab, ascomycin, -estradiol, rosiglitazone, troglitazone, pioglitazone, S-nitrosoglutathione, gliotoxin G, panepoxy done, cycloepoxydon tepoxalin.

In one embodiment of this invention, a composition includes at least one angiogenesis inhibitor and at least one analgesic compound. Examples of analgesic compounds include bupivacaine, lidocaine, ropivacaines, and gabapentin.

One embodiment composition includes at least one angiogenesis inhibitor and at least one injectable osteoarthritis treatment agent, most preferably a viscosupplement. The viscosupplement can be hyaluronic acid.

In another embodiment, a composition includes at least one angiogenesis inhibitor, a viscosupplement and a hydrophobic sugar. The hydrophobic sugar is can include sucro acetate isobutyrate (SAIB).

In yet another embodiment, a composition includes at least one angiogenesis inhibitor, a viscosupplement, a hydrophobic sugar and a therapeutic solvent. The solvent can include Dimethysulfoxide (DMSO).

In some of its various embodiments, the present invention can provide several treatment modalities to users. Treatment may include the administration of an effective amount of at least one angiogenesis inhibitor, preferably in a pharmaceutically acceptable composition that contains at least one such compound. Alternatively, treatment may include administration of at least one angiogenesis inhibitor in combination with administration of at least one other anti-inflammatory treatment agent, preferably in a composition containing both the angiogenesis inhibitor and the other anti-inflammatory treatment agent.

Some formulation embodiments include a viscosupplement, a therapeutic solvent, a hydrophobic sugar and one or more angiogenesis inhibitors. In some embodiments, the viscosupplement includes hyaluronic acid. The hydrophobic sugar is also a viscosupplement in some embodiments. The formulation includes drug particles in some embodiments. The angiogenesis inhibitor can be in the form of micro or nano particulates in some embodiments, where the micro or nano particulates can include polymer providing controlled rates of drug release in the region to be treated. The hydrophobic sugar includes sucro acetate isobutyrate (SA18) in some formulations. In some embodiments, the therapeutic solvent includes dimethyl sulfoxide (DMSO), ethyl acetate, vinyl-pyrrolidinone, and/or ethanol and combinations thereof.

The angiogenesis inhibitor can include an mTor inhibitor in a dosage effective for treatment of osteoarthritis in the region of the joint where administered, wherein the mTor inhibitor can be rapamycin, rapamycin analogs, curcumin and combination thereof in various embodiments. The angiogenesis inhibitor can include an anti-VEGF agent effective for treatment of osteoarthritis in the region of the joint where administered. The anti-VEGF agent can include endostatin, angiostatin, canstatin, combrestatin, contortrostatin, fumagillin, TNP-470, catechin, paclitaxel, rapamycin and rapamycin analogs, neovastat, 2-methoxy estradiol, and mixtures thereof in various embodiments.

In some embodiments, the drug includes an anti-bFGF agent effective for treatment of osteoarthritis in the region of the joint where administered. The anti-bFGF agent can include interferon-a and platelet factor 4, halofuginone, tranilast, 2-methoxyestradiol, Imatinib (Glivec), Trastuzumab (Herceptin), Celebrex and mixtures thereof in various embodiments. In some embodiments, the angiogenesis inhibitor can include a MAPK inhibitor effective for treatment of osteoarthritis in the region of the joint where administered. The MAPK inhibitor can include curcumin, 58203580, 58239063, SFK-86002, 5-lodotubercidin, Apigenin, Arctigenin and mixtures thereof in various embodiments.

In some embodiments of the invention, the angiogenesis inhibitor is administered in combination with a NF-KB inhibitor effective for treatment of osteoarthritis in the region of the joint where administered. The NF-KB inhibitor can include curcumin, sulfasalazine, sulindac, indomethacin, diclofenal, etodolac, meclofenate, mefenamic acid, nambunetone, piroxicam, phenylbutazone, meloxicam, dexamethasone, betamethasone dipropionate, diflorsasone diacetate, clobetasol propionate, halobetasol propionate, amcinomide, beclomethasone dipropionate, fluocinomide, betamethasone valerate, triamcinolone acetonide, penicillamine, hydroxychloroquine, sulfasalazine, azathioprine, minocycline, cyclophosphamide, methotrexate, cyclosporine, leflunomide, etanercept, infliximab, ascomycin, beta-estradiol, rosiglitazone, troglitazone, pioglitazone, S-nitrosoglutathione, gliotoxin G, panepoxydone, and cycloepoxydon tepoxalin and mixtures thereof in various embodiments.

In some embodiment formulations the angiogenesis inhibitor is pegylated. The pegylated angiogenesis inhibitor includes pegylated Interferon-a in some embodiments. The present patent application also provides methods for treating osteoarthritis including placing into a joint one or more of the formulations described in the present application, including those above, for example, by intra-articular injection. In some methods, the concentration of the angiogenesis inhibitor is between 0.0001-10 mg/ml.

Some ingredients in the formulation can serve more than one function. Curcumin and rapamycin can serve as AA, Al, AIS or AAS, depending on what it is intended to inhibit. For example, if rapamycin is used as an Al then curcumin can be used as an Al or AIS/AAS and vice versa, and more curcumin and less rapamycin could be used in the formulation. MMP inhibitors can function as anti-inflammatory agents, as MMPs help to regulate the inflammation process.

In joints having already formed blood vessels and nerves, some embodiments of the formulation can act to prevent inflammation and potential angiogenesis as a result of the inflammation. In some embodiment methods, blood vessels and nerves can be killed, for example, using laser or cyrotherapy or drugs. One of the formulations can then be applied.

DMSO can have more than one function in the injectable formulation. It can function as: a drug as an anti-inflammatory drug; a solvent to dissolve a drug or drugs; a thinning solvent to dissolve or disperse the hydrophobic sugar; a nano or microparticle forming solvent; a sensitizer or a permeation enhancer as a cell membrane penetrant.

Other form of polymers (for example slow degradable polymers or biostable polymers) can be used as well in place of the hydrophobic sugar, in some embodiments. Some of the examples include PLLA, PCL, Polyurethanes, PLGA.

The present patent application provides methods for treating osteoarthritis in a joint having an intra-articular space and tissue near the intra-articular space. The method can include injecting a plurality of hydrophobic nano-particles into the intra-articular space. The nano-particles are substantially spherical in some embodiments, and have a major dimension of less than about 100 microns, 50 microns, and 10 microns, in the various embodiments. The nano-particles can include an anti-inflammatory agent (AA) and an angiogenesis inhibitor (Al), where the nano-particles are configured to controllably release the AA and the Al over time. The AA and the Al can be present together within the same nano-particles or in separate nano-particles, in various embodiments. In some embodiments, the nano-particles are configured to release the AA and Al through diffusion substantially before substantial nano-particle structure degradation.

Some embodiment methods also deliver an anti-inflammatory agent sensitizer (AAS) that increases the effectiveness of the AA by at least 4 times. Some embodiment methods also deliver an angiogenesis inhibitor sensitizer (AIS) that increases the effectiveness of the Al by at least 4 times. Methods can also deliver an anti-inflammatory agent sensitizer (AAS) which increases the effectiveness of the AA by at least 4 times, and an angiogenesis inhibitor sensitizer (AIS) which increases the effectiveness of the Al by at least 4 times.

Some methods deliver nano-particles, in which the nano-particles include a first set of nano-particles having a first size and a first time to deliver about half of the finally delivered AA and a second set of nano-particles having a second size and a second time to deliver about half of the finally delivered AA, wherein the second size is substantially larger than the first size and the second time is substantially larger than the first time. In some methods, there is at least about 5 times more AA delivered than Al by weight. In various embodiment methods, the nano-particles are configured to deliver the AA and the Al in therapeutically significant amounts over at least about 1 week, 2 weeks, 1 month, or 2 months, depending on the embodiment. Some methods also deliver a viscosupplement.

In some methods, the AA includes a steroid and the Al includes rapamycin. The AIS can include curcumin and the AAS can include sulfazine. In some methods, at least one of the AA or the Al is an mTor inhibitor.

Some embodiments of the present invention provide methods for treating osteoarthritis in a joint having an intra-articular space and tissue near the intra-articular space. The methods can include delivering a therapeutically effective amount of an anti-inflammatory agent (AA) and a therapeutically effective amount of an angiogenesis inhibitor (Al) into the intra-articular space. In some methods, the AA and the Al are delivered from an injected controlled release carrier, which can include the AA and the Al being controllably released from nano-particles, in various embodiments. The AA and the Al can be controllably released through a diffusion-controlled mechanism in some methods. In some embodiments, the AA and the Al are controllably released through degradation of the nano-particles. In some methods, the nano-particles include hydrophobic nano-particles, which can include one or more hydrophobic sugars.

In some methods the AA and the Al are delivered through a tube having a lumen within and a first tube region placed to deliver the AA and the Al to the intra-articular space and a second tube region placed in communication with an AA and Al supply reservoir located outside of the intra-articular space. In some embodiments, the reservoir can include a subcutaneous reservoir replenishable through injection. Osmotic and mechanical pumps can be used in some methods.

Some methods also deliver an anti-inflammatory agent sensitizer (AAS) which increases the effectiveness of the AA by at least 4 times and/or an angiogenesis inhibitor sensitizer (AIS) which increases the effectiveness of the Al by at least 4 times. At least one of the AA or the Al is an mTor inhibitor in some methods.

Some embodiments of the present invention provide compositions for treating osteoarthritis in a joint having an intra-articular space and tissue near the intra-articular space. The composition can include a plurality of hydrophobic nano-particles having a major dimension of less than about 100 microns, in which the nano-particles include an anti-inflammatory agent (AA) and an angiogenesis inhibitor (Al) and in which the nano-particles are configured to controllably release the AA and the Al over time. The compositions can include nano-particles having a major dimension of less than about 50, 20, and 10 microns, in various embodiments. The AA and the Al are present together within the same nano-particles in some embodiments, and are present in separate nano-particles in other embodiments. The nano-particles are configured to release the AA and Al through diffusion substantially before substantial nano-particle structure degradation in some embodiments, and are configured to release the AA and Al through substantial nano-particle structure degradation in other embodiments.

Some compositions also include an anti-inflammatory agent sensitizer (AAS) which increases the effectiveness of the AA by at least 4 times and/or an angiogenesis inhibitor sensitizer (AIS) which increases the effectiveness of the Al by at least 4 times.

In some composition embodiments, the nano-particles include a first set of nano-particles having a first size and a first time to deliver about half of the finally delivered AA and a second set of nano-particles having a second size and a second time to deliver about half of the finally delivered AA, wherein the second size is substantially larger than the first size and the second time is substantially larger than the first time. In some composition embodiments, the nano-particles include a first set of nano-particles having a first size and a first time to deliver about half of the finally delivered Al and a second set of nano-particles having a second size and a second time to deliver about half of the finally delivered Al, wherein the second size is substantially larger than the first size and the second time is substantially larger than the first time.

In some composition embodiments, there is at least about 5 times more AA present than Al by weight. The nano-particles are configured to deliver the AA and the Al in therapeutically significant amounts over at least about 1 week, 2 weeks, 1 month, or 2 months, in various embodiments. Some compositions include a viscosupplement. In some compositions, the AA includes a steroid and the Al includes rapamycin. The AIS includes curcumin and the AAS includes sulfazine in some embodiments. At least one of the AA or the Al is an mTor inhibitor in some compositions.

### BRIEF DESCRIPTION OF THE DRAWINGS

FIG. 1 depicts an injectable formulation of sulfasalazine in sucrose acetate isobutyrate (SAIB) and DMSO.

FIG. 2 depicts the injectable formulation of FIG. 1 when injected into an aqueous environment.

FIG. 3 is a drawing of a nano-particle solution of curcumin (an antiangiogenesis, mTor inhibitor and anti-inflammatory drug) encapsulated in SAIB gel and suspended in phosphate buffer saline (PBS).

FIG. 4 is graphical representation of the measured levels of rapamycin (y-axis, cumulative micrograms) released over time (x-axis, days) in sterile PBS at 37 degrees C. from two formulations of SAIB/rapamycin nanoparticles suspended in a solution of hyaluronic acid with rapamycin encapsulated within the SAIB material.

FIG. 5 is graphical representation of the measured levels of curcumin (y-axis, cumulative micrograms) released over time (x-axis, days) in sterile PBS at 37 degrees C. from a formulation of SAIB/curcumin nanoparticles suspended in a solution of hyaluronic acid with curcumin encapsulated within the SAIB material. The elution curve indicated that drug could be released over an extended period of time.

FIG. 6 is graphical representation of the measured levels of sulfasalazine (y-axis, cumulative micrograms) released over time (x-axis, days) in sterile PBS at 37 degrees C. from a formulation of SAIB/sulfasalazine nanoparticles suspended in a solution of hyaluronic acid with sulfasalazine encapsulated within the SAIB material.

### DETAILED DESCRIPTION OF THE INVENTION

The present invention includes methods and compositions wherein angiogenesis inhibitors are used to treat the abnormal angiogenesis related to inflammation, pain and tissue destruction associated with osteoarthritis.

Angiogenesis and chronic inflammation are closely integrated processes. Inflammation can stimulate angiogenesis, and angiogenesis can facilitate inflammation. However, although chronic inflammation is almost always accompanied by angiogenesis, angiogenesis can occur in the absence of inflammation.

It is believed, without being bound by theory, that the mechanism responsible for the beneficial effects of treating osteoarthritis with angiogenesis inhibitors in accordance with the present invention involves their ability to inhibit the angiogenesis process that can facilitate inflammation, which if unchecked, leads to elevated levels of MMPs that can leads to the chronic inflammation, cartilage destruction, pain and loss of joint mobility associated with osteoarthritis.

Specific examples of angiogenesis inhibitors include, but are not limited to, mTor inhibitors, anti-VEGF, anti-bFGF, and mitogen-activated protein kinase (MAPK) inhibitors.

Representative examples of mTor inhibitors include rapamycin, rapamycin analogs (Temsirolimus, 8iolimus, Everolimus, Zotarolimus or A8T-578), curcumin, and mixtures thereof.

Representative examples of anti-VEGF include endostatin, angiostatin, canstatin, combrestatin, contortrostatin, fumagillin, TNP-470, catechin, paclitaxel, rapamycin and rapamycin analogs, neovastat, 2-methoxy estradiol, and mixture thereof.

Representative examples of anti-bFGF include interferon-a and platelet factor 4, halofuginone, tranilast, methoxyestradiol, Imatinib (Glivec), Trastuzumab (Herceptin), Celebrex and mixture thereof.

Representative examples of MAPK inhibitors include curcumin, 58203580, 58239063, SFK-86002, 5-lodotubercidin, Apigenin, Arctigenin and mixtures thereof.

In one embodiment of the present invention, a method for treating osteoarthritis comprises directly administering to the joint an effective amount of at least one angiogenesis inhibitor. Administering the angiogenesis inhibitor can be accomplished by intra-articular injection of a composition comprising at least one angiogenesis inhibitor into an arthritic joint. Intra-articular injection differs from other methods of administering medication in that it allows biologically sufficient concentrations of active pharmaceutical ingredient to be applied to the affected synovial tissue without the risk of producing the undesirable side-effects that can occur as the result of the higher concentrations of drug required by other administration techniques.

In one embodiment of the invention, an effective amount of one or more angiogenesis inhibitors is administered to an osteoarthritic joint in a pharmaceutically acceptable composition that is sufficient to reduce any or all of the symptoms of osteoarthritis in the treated joint, such as inflammation, pain, stiffness and/or loss of function, without producing any of the undesirable side effects resulting from an overdose of angiogenesis inhibitors, such as tissue death or injury, joint swelling, etc.

An effective amount of angiogenesis inhibitors for treating osteoarthritis in accordance with the present invention using intra-articular injection may be in the range of 0.00001-10.0 mg, for example, in the form of nanoparticles or microparticles suspended in physiological saline or other vehicle appropriate for injection into the body. Some compositions include one or more angiogenesis inhibitors at a total concentration of 0.00001-10.0 mg/ml.

In another embodiment of the invention, one or more angiogenesis inhibitors may be administered in combination with one or more other inflammatory treatment agents in the same composition preferably in the form of an injectable composition, i.e. a composition that is suitable for being injected directly into the affected joint (intra-articular injection). The treatment method of the present invention can readily be customized to the individual Patient's needs based on the severity of the disease.

In another embodiment of the invention, one or more angiogenesis inhibitors may be co-dissolved with a hydrophobic sugar in a solvent that is suitable for injection into the joint. The hydrophobic sugar can include sucro acetate isobutyrate (SAIB). The solvent in this invention can be dimethyl sulfoxide (DMSO), ethanol, n-vinylpyrrolidone or ethyl acetate. In one embodiment, concentration of the hydrophobic sugar in the composition is between 5-150 mg/ml.

In one embodiment of the invention, a mixture of a hydrophobic sugar and at least one or more angiogenesis inhibitors is mixed with a viscosupplement solution to form nanoparticles or microparticles that encapsulated the angiogenesis inhibitor. The nanoparticles or microparticles allow sustained release of the drugs and prevent overdosing and toxicity in the region of treatment.

In another embodiment of the invention, the ratio of the hydrophobic sugar and the viscosupplement in the injectable formulation is varied between 1:1 to 1:20 and preferably between 1:5 to 1:10.

In still another embodiment of the invention, the mixture of hydrophobic sugar includes at least one or more angiogenesis inhibitors and at least one or more anti-inflammatory agents.

Examples of anti-inflammatory agents include curcumin, sulfasalazine, sulindac, indomethacin, diclofenac, etodolac, meclofenate, mefenamic acid, nambunetone, piroxicam, phenylbutazone, meloxicam, dexamethasone, betamethasone dipropionate, diflorsasone diacetate, clobetasol propionate, halobetasol propionate, amcinomide, beclomethasone dipropionate, fluocinomide, betamethasone valerate, triamcinolone acetonide, penicillamine, hydroxychloroquine, sulfasalazine, azathioprine, minocycline, doxycycline, cyclophosphamide, methotrexate, cyclosporine, leflunomide, etanercept, infliximab, ascomycin, beta-estradiol, rosiglitazone, troglitazone, pioglitazone, S-nitrosoglutathione, gliotoxin G, panepoxydone, cycloepoxydon tepoxalin and mixtures thereof.

The hydrophobic sugar can be used as a viscosupplement to restore and/or increase the cushioning and lubrication of arthritic synovial fluid by intra-articular injection. The hydrophobic sugar may lengthen the treatment period without the need for crosslinking as in the case of other currently available products such as Synvisc® and Hyalgan®.

In one embodiment of the invention, the injectable mixture of one or more angiogenesis inhibitors includes small amounts of vitamin D, magnesium, zinc or manganese. The concentration of these agents can be between 0.00001 mg-0.5 mg/ml of injectable formulation.

In another embodiment of the invention, the angiogenesis inhibitors in the injectable mixture are pegylated in order to improve bioavailability of the drug and prolong their release profiles. An example of pegylated drug is pegylated interferon (a and). Different molecular weight (MW) of polyethylene glycol (PEG) can be used to in the pegylation of the angiogenesis inhibitor depending on the timing of drug delivery and the chemical characteristic of the angiogenesis inhibitor. Pegylation of drugs will be apparent to those skilled in the art.

Accordingly, examples of treatments contemplated by the present invention include an intra-articular injection of a composition including one or more angiogenesis inhibitors in a non-aqueous solution, an intra-articular injection of an aqueous solution containing nanospheres or microspheres of one or more angiogenesis inhibitors encapsulated in a hydrophobic sugar, or an intraarticular injection of a single composition comprising at least one angiogenesis inhibitor and at least one viscosupplement, steroid or other anti-inflammatory agent; and so forth.

A treatment composition according to one embodiment of the invention comprises one or more angiogenesis inhibitor(s) and one or more anti-inflammatory agent(s). The individual concentrations of the angiogenesis inhibitors(s) and the anti-inflammatory agent(s) are sufficient to provide an effective amount of each ingredient to the affected joint. The composition can include angiogenesis inhibitors(s) at a concentration of 0.00001-10.0 mg/ml and other anti-inflammatory agent(s) at a concentration of 0.001-25 mg/ml.

In one embodiment, the composition is suitable for intra-articular injection in accordance with the method of the present invention, and both the angiogenesis inhibitor and inflammatory treatment agent are injectable. As used herein, the term “injectable” means any inflammatory treatment agent that is in a form suitable for intra-articular injection.

In another embodiment, the injectable inflammatory treatment agents may include at least one NF-KB inhibitor. As one specific non-limiting example, the composition of the present invention may comprise 0.1-10 mg/ml of the injectable NF-KB inhibitor sulfasalazine or curcumin.

In still another embodiment, the injectable formulation may include at least one viscosupplement. As used herein and in the art, the term “viscosupplement” refers to any substance that is used to restore and/or increase the cushioning and lubrication of arthritic synovial fluid by intra-articular injection. Preferred viscosupplements include hylan, hyaluronic acid and other hyaluronan (sodium hyaluronate) compounds, which are natural complex sugars of the glycosaminoglycan family. Hyaluronan, in particular, is a long-chain polymer containing repeating disaccharide units of Na-glucoronate-N-acetylglucosamine. Examples of commercially available hyaluronan viscosupplements include Synvisc®, Hyalgan®, Supartz®, and Orthovisc®.

As one specific non-limiting example, the composition of the present invention may include 5-15 mg/ml of hyaluronic acid.

The formulations of the present invention may also contain other materials such as surfactants, fillers, stabilizers, coloring agents, preservatives, and other additives known in the art. The formulations may be in liquid or gel form and may be provided in time-release formulations.

It is common for patients with osteoarthritis or joint injuries to under go arthroscopic surgical procedures. These procedures are common for the knee, shoulder, elbow and other large joints. Common treatments with knee arthroscopy include: (1) removal or repair of torn meniscal cartilage, (2) reconstruction of a torn cruciate ligament, (3) trimming of torn pieces of articular cartilage, (4) removal of loose fragments of bone or cartilage and (5) removal of inflamed synovial tissue. Following these treatments, it might be desirable to deliver the formulation disclosed in this invention. The formulation can be tailored for arthroscopic delivery. This may include increasing the viscosity of the formulation, formation into a sheet or disk. This formulation may not be deliverable through a needle.

The high viscosity formulation can be delivered with a device consisting of a tubular member with an outer diameter sufficient to be passed through a port, trocar, or incision appropriate for arthroscopic procedures. The formulation can be loaded into the bore of the tubular member. The length of the device is sufficient to pass through a port, trocar, or incision while reaching the appropriate anatomical location and extending outside the body sufficiently to allow the medical professional to direct the distal delivery.

Within the bore of the device and distal to the loaded formulation there can be a plunger. Attached to the plunger is a plunger actuator that extends beyond the length of the tubular member. Upon locating the distal orifice at the desired spot of delivery the medical professional can depress the plunger actuator forward and dispense the therapeutic formulation.

In some instances it may be desirable to have the formulation in a pre-made form. These forms may include a trimmable sheet, plug, capsule, etc. In these cases the therapeutic compound maybe incorporated into a collagen matrix. The collagen matrix form can be rolled and placed inside the delivery device for passing through the port, trocar or incision.

During an arthroscopic joint procedure, a sterile solution can be infused in the joint to expand the soft tissues to provide a work zone. Either or both the therapeutic compound or the sterile solution can be tailored so that they do not adversely affect the other. A common sterile solution is 0.7% saline. The therapeutic compound and or therapeutic matrix can be compounded to provide a hydrophobic state adequate for the duration of exposed to the saline.

### Formulations

The formulations can be designed to provide maximum uptake in the affected tissues with rapid dissemination throughout the region to be treated, with little to no increase in systemic blood levels of the drug. The formulations can consist solely of drug, or drug combined with excipient or polymeric material.

### Drugs

The term “drug” can refer to any pharmaceutically active substance capable of being administered in a particulate formulation, which achieves the desired effect. Drugs can be synthetic or isolated natural organic compounds, proteins or peptides, oligonucleotides or nucleotides, or polysaccharides or sugars. Drugs may have any of a variety of activities, which may be inhibitory or stimulatory, such as antibiotic activity, antiviral activity, antifungal activity, steroidal activity, cytotoxic or anti-proliferative activity, anti-inflammatory activity, analgesic or anesthetic activity, as well as contrast or other diagnostic agents.

### Excipients or Carriers

The drug substance may be “associated” in any physical form with a particulate material, for example, adsorbed or absorbed, adhered to or dispersed or suspended in such matter, which may take the form of discrete particles or microparticles in any medicinal preparation, and/or suspended or dissolved in a carrier such as an ointment, gel, paste, lotion, or spray.

Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars and starches.

In one embodiment of this invention, the excipient is a hydrophobic sugar such as sucro acetate isobutyrate (SAIB).

In one embodiment, the drug is present on or within micro or nanoparticulates formed of a polymeric material. Polymers can be used to control release as a function of the diffusion rate of drugs out of the polymeric matrix and/or rate of degradation by hydrolysis or enzyme degradation of the polymers and/or pH alteration, and to increase surface area of the drug relative to the size of the particle.

### Polymeric Materials

Generally, the classes of polymers which applicants believe to have useful properties for use as viscosupplement are hydrophilic natural polymers and hydrogels. In the class of hydrophilic natural polymers, those containing disaccharide-repeating unit (e.g., hyaluronic acid) exhibit good properties for intra-articular injection.

Rapidly bio-erodible polymers such as poly [lactide-co-glycolide], polyanhydrides, and polyorthoesters, whose carboxylic groups are exposed on the external surface as their smooth surface erodes, are good candidates for drug delivery systems. In addition, polymers containing labile bonds, such as polyanhydrides and polyesters, are well known for their hydrolytic reactivity. Their hydrolytic degradation rates can generally be altered by simple changes in the polymer backbone.

Representative natural polymers include proteins, such as zein, modified zein, casein, gelatin, gluten, serum albumin, or collagen, and polysaccharides, such as cellulose, dextrans, polyhyaluronic acid, polymers of acrylic and methacrylic esters and alginic acid. Representative synthetic polymers include polyphosphazines, poly (vinyl alcohols), polyamides, polycarbonates, polyalkylenes, polyacrylamides, polyalkylene glycols, polyalkylene oxides, poly alkylene terephthalates, polyvinyl ethers, polyvinyl esters, polyvinyl halides, polyvinylpyrrolidone, polyglycolides, polysiloxanes, polyurethanes and copolymers thereof.

Synthetically modified natural polymers include alkyl celluloses, hydroxyalkyl celluloses, cellulose ethers, cellulose esters, and nitrocelluloses. Other polymers of interest include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, and polyvinylphenol. Representative bio-erodible polymers include polylactides, polyglycolides and copolymers thereof, poly(ethylene terephthalate), poly(butic acid), poly(valeric acid), poly(lactide-co-caprolactone), poly[lactide-co-glycolide], polyanhydrides, polyorthoesters, blends and copolymers thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-co-caprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

These polymers can be obtained from sources such as Sigma Chemical Co., St. Louis, Mo., Poly sciences, Warrenton, Pa., Aldrich, Milwaukee, Wis., Fluka, Ronkonkoma, N.Y., and BioRad, Richmond, Calif. or else synthesized from monomers obtained from these suppliers using standard techniques. Both non-biodegradable and biodegradable matrices can be used for delivery of drugs, although biodegradable matrices are preferred. These may be natural or synthetic polymers, although synthetic polymers are preferred due to the better characterization of degradation and release profiles. The polymer is selected based on the period over which release is desired, generally in the range of at least immediate release to release over a period of twelve months, although longer periods may be desirable. In some cases linear release may be most useful, although in others a pulse release or “bulk release” may provide more effective results. The polymer may be in the form of a hydrogel (typically absorbing up to about 90% by weight of water), and can optionally be cross-linked with multivalent ions or polymers.

High molecular weight drugs can be delivered partially by diffusion but mainly by degradation of the polymeric system. In this case, biodegradable polymers, bio-erodible hydrogels, and protein delivery systems are particularly preferred.

Examples of non-biodegradable polymers include ethylene vinyl acetate, poly(meth)acrylic acid, polyamides, copolymers and mixtures thereof.

### EXAMPLES

### Materials and Methods

Curcumin (Cat. #C1386), Sulfasalazine (Cat. #S0883), Hyaluronic acid sodium salt from rooster comb (Cat. #H5388), Rapamycin from Streptomyces hygroscopicus (Cat. #R0395), dimethyl sulfoxide (Cat. #D8418), Sucrose acetate isobutyrate solution (Cat. #525286) and phosphate buffered saline powder (Cat. #P3813) were available from Sigma, St. Louis, Mo. All other chemicals were from Fisher Scientific unless otherwise noted. Dialysis tubing (Cat. #D0530, high retention seamless cellulose tubing) was also available from Sigma, St. Louis, Mo. All percentages are based on weight unless otherwise noted.

### Example 1

### Preparation of an Injectable Formulation of Sulfasalazine in SAIB Gel

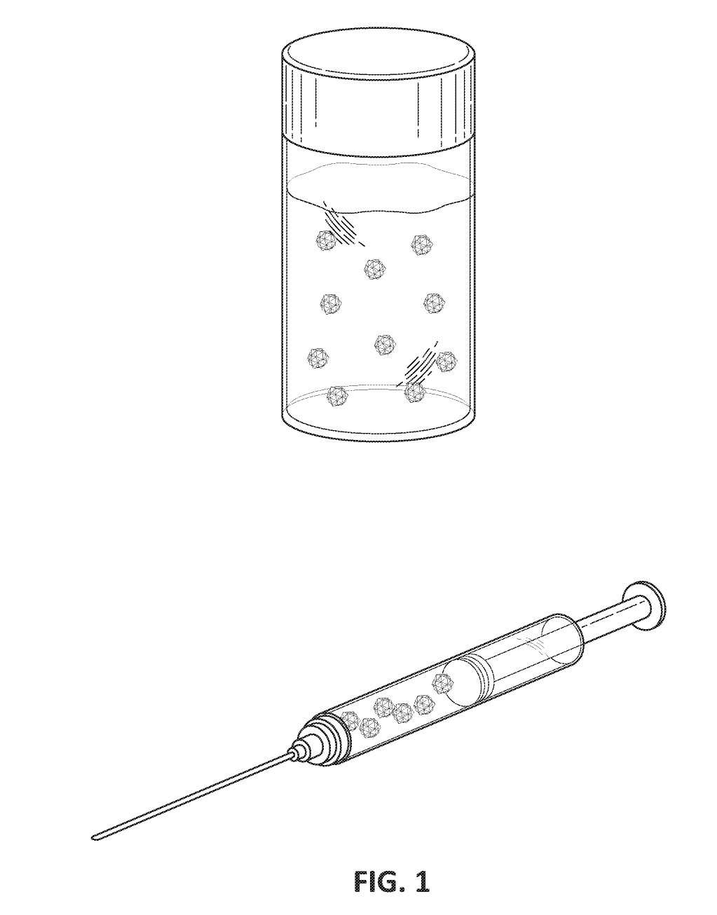

FIG. 1 is a drawing of an injectable formulation of sulfasalazine in sucrose acetate isobutyrate (SAIB) and DMSO to form a less viscous solution for injection into the affected joint. Sulfasalazine is an anti-inflammatory agent and also is a NF-kB inhibitor.

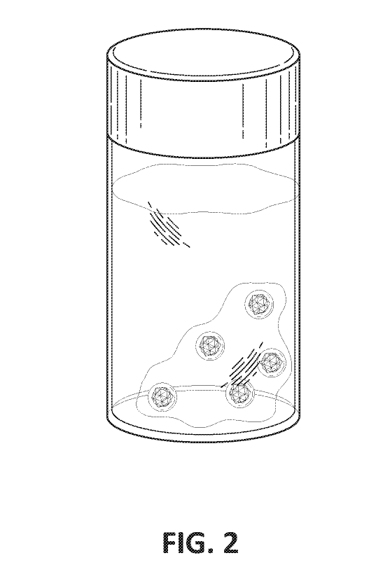

FIG. 2 is a drawing of the injectable formulation of FIG. 1 when injected into an aqueous environment. The liquid formulation of drug, DMSO and SAIB formed a liquid gel after the ethanol component in the SAIB and DMSO diffused out into the water. The drug, in this case, sulfasalazine slowly diffused out of the gel in a controlled release manner. Controlled release of the drug over a long period of time can be beneficial to treat chronic inflammation in osteoarthritis.

A known amount of sulfasalazine was weighed and dissolved in DMSO. This solution of sulfasalazine/DMSO was then mixed in 1:1 ratio with SAIB gel (90% by weight in ethanol). The mixture as shown in FIG. 1 can be loaded into a syringe and injected through a 20 GA needle.

The mixture formed a gel that encapsulated the drug after the solvents (DMSO and ethanol) diffused out of the aqueous environment (FIG. 2). The drug can then be released in a controlled release manner to treat the disease.

### Example 2

### Preparation of Nanoparticles of Curcumin and Injectable Formulation

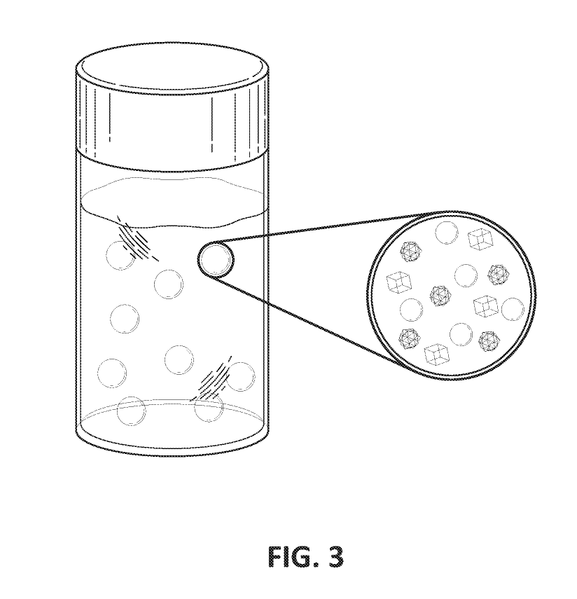

FIG. 3 is a drawing of a nano-particle solution of curcumin (an antiangiogenesis, mTor inhibitor and anti-inflammatory drug) encapsulated in SAIB gel and suspended in phosphate buffer saline (PBS).

About 21 mg of curcumin was dissolved in 2 ml of DMSO/SAIB (9:1 ratio of DMSO to SAIB in ethanol). The concentration of SAIB in the solution was about 104 mg per ml of DMSO/SAIB/Ethanol solution. This solution of curcumin/DMSO/SAIB/Ethanol was added drop wise to PBS while stirring. The nanoparticles of curcumin/SAIB formed when the DMSO/ethanol diffused out into the PBS (FIG. 3). 0.5 ml of the nanoparticle solution was then mixed with 2.5 ml of 1% Hyaluronic acid (HA) in PBS. The final mixture was then used for testing of injectability and for elution study.

### Example 3

### In Vitro Release of Various Injectable Formulations

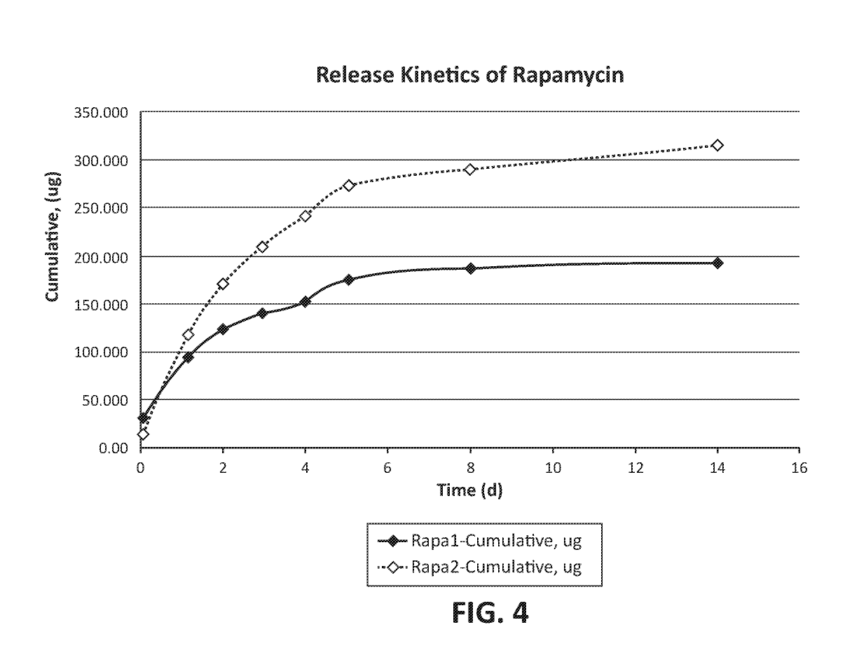

FIG. 4 is graphical representation of the measured levels of rapamycin released over time from two formulations of rapamycin encapsulated within SAIB nanoparticles suspended in a solution of hyaluronic acid.

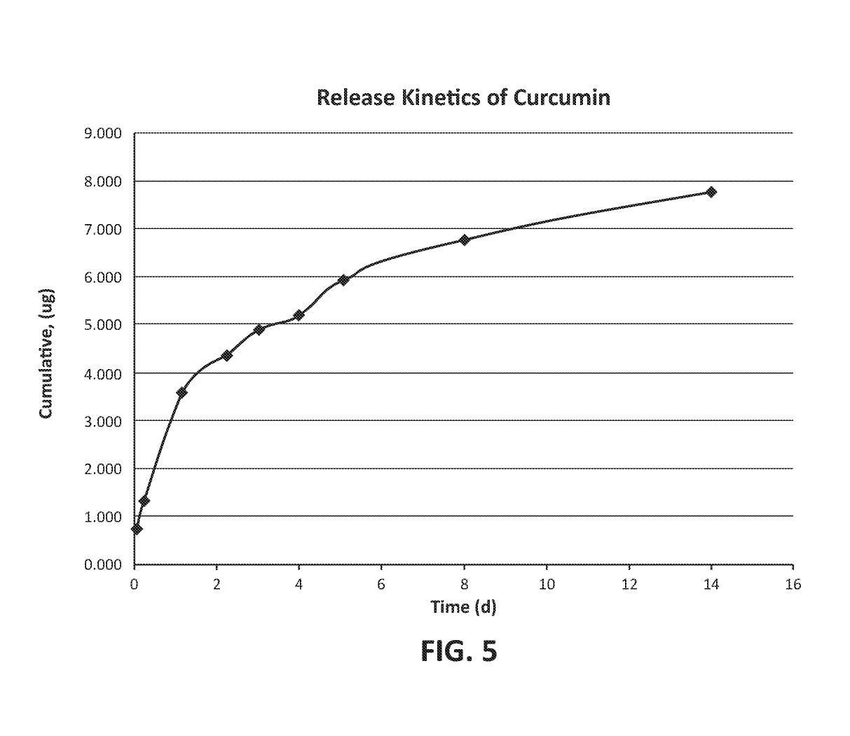

FIG. 5 is graphical representation of the measured levels of curcumin released over time from a formulation of curcumin encapsulated within SAIB nanoparticles suspended in a solution of hyaluronic acid. The elution curve indicated that drug can be released over an extended period of time.

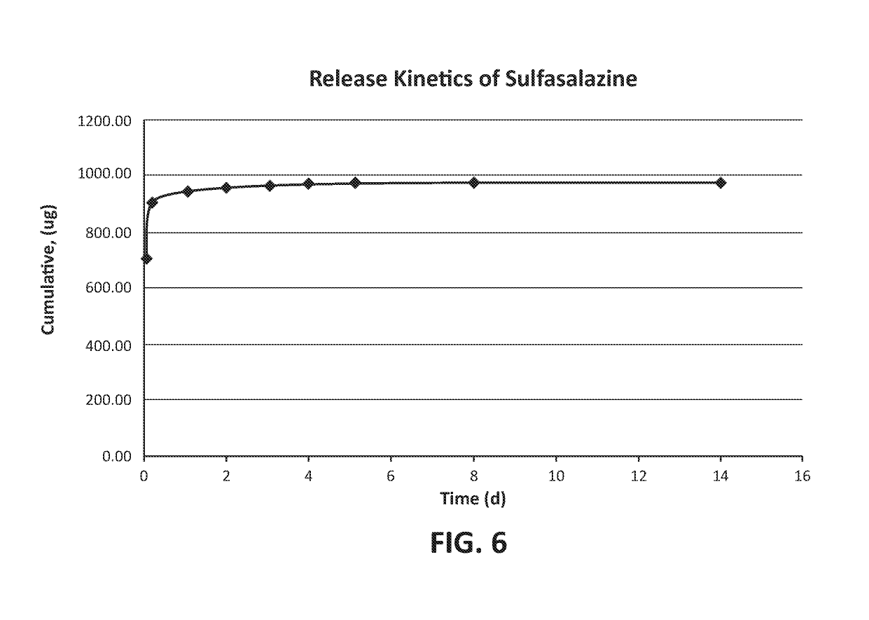

FIG. 6 is graphical representation of the measured levels of sulfasalazine released over time from a formulation sulfasalazine encapsulated within SAIB nanoparticles suspended in a solution of hyaluronic acid.

Elution testing of various injectable formulations as described in example 2 was carried out in sterile PBS and at 37 degrees C. Drugs such as rapamycin, curcumin and sulfasalazine were used in the testing of various formulations. Each formulation was pipetted into a segment of dialysis tubing (Sigma Cat. D0530) with both ends of the tubing sealed. The dialysis tubing with the formulation was then placed inside a container and 15 ml of sterile PBS was added to the container. The container was then placed in an incubator shaker at 37 degrees C., 50 rpm and samples were withdrawn at regular time intervals and replaced with fresh sterile PBS. The samples were measured photometrically (UV-Vis) at a wavelength of 420 nanometers for curcumin, 359 nanometers for sulfasalazine and 278 nanometers for rapamycin.

The release kinetics of rapamycin from two formulations with different amount of hyaluronic acid are shown in FIG. 4. The levels of released rapamycin measured is dependent on the amount of hydro gel in the solution of the injectable formulation. The release kinetics of curcumin from a similar formulation shows that curcumin release from the nanoparticles in a controlled release manner (FIG. 5). In the case of sulfasalazine, since the drug is slightly water soluble, the release kinetics is high at the beginning and level off quickly in a few days.

In some methods of manufacture, at two miscible solvents (for example DMSO and ethanol) and a hydrophobic sugar (for example SAIB) are used to create a nano-particle solution containing at least one drug. The ratio of the two solvents (DMSO and ethanol) are in at least about a 9:1 ratio and the concentration of the sugar is less than about 10% by weight, in some embodiments. The nano-particle solution can then be sterilized and filtered. A hydro gel solution in PBS having a concentration of about 1% or less can be aseptically formed. The hydrogel solution can be a hyaluronic acid solution. The nano-particle solution can be mixed with the hydrogel solution in about a 1:5 to 1:1 ratio. This mixture can then be loaded into a syringe or other deliver device and delivered into an osteoarthritic joint.

1. A method for treating osteoarthritis in a joint having an intra-articular space and tissue near the intra-articular space, the method comprising:
delivering a therapeutically effective amount of an anti-inflammatory agent (AA) and a therapeutically effective amount of an angiogenesis inhibitor (Al) into the intra-articular space.

2. The method of claim 1, in which the AA and the Al are delivered from one or more controlled release carriers.

3. The method of claim 2, in which the AA and the Al are controllably released from nanoparticles.

4. The method of claim 3, in which the AA and the Al are controllably released through diffusion controlled mechanism.

5. The method of claim 3, in which the AA and the Al are controllably released through degradation of the nano-particles.

6. The method of claim 3, in which the nano-particles include hydrophobic nano-particles.

7. The method of claim 6, in which the nano-particles include hydrophobic sugar.

8. The method of claim 1, in which the AA and the Al are delivered through a tube having a lumen within, a first region placed to deliver the AA and the Al to the intra-articular space, and a second region placed in communication with an AA and Al supply reservoir located outside of the intra-articular space.

9. The method of claim 1, further comprising an anti-inflammatory agent sensitizer (AAS) which increases the effectiveness of the AA.

10. The method of claim 1, further comprising an angiogenesis inhibitor sensitizer (AIS) that increases the effectiveness of the Al.

11. The method of claim 1, further comprising an anti-inflammatory agent sensitizer (AAS) which increases the effectiveness of the AA by at least 4 times, and an angiogenesis inhibitor sensitizer (AIS) which increases the effectiveness of the Al by at least 4 times.

12. The method of claim 1, in which at least one of the AA or the Al is an mTor inhibitor.

13. A method for treating osteoarthritis in a joint having an intra-articular space and tissue near the intra-articular space, the method comprising:
injecting a plurality of hydrophobic nano-particles into the intra-articular space.

14. The method of claim 13 where the nano-particles are substantially spherical.

15. The method of claim 13, where the nano-particles have a major dimension of less than about 100 microns.

16. The method of claim 13, where the nano-particles have a major dimension of less than about 50 microns.

17. The method of claim 13, in which the nano-particles include an anti-inflammatory agent (AA) and an angiogenesis inhibitor (Al) and in which the nano-particles are configured to controllably release the AA and the Al over time.

18. A method for treating injury in a joint having an intra-articular space and tissue near the intra-articular space by injecting a viscous solution consisting of:
a plurality of hydrophobic nano-particles forming from mixing an aqueous solution of viscosupplement and a solution of a hydrophobic polymer comprising of two or more organic solvents and two or more pharmaceutical agents;
in which the nano-particles include an anti-inflammatory agent (AA) and an angiogenesis inhibitor (Al) and in which the nano-particles are configured to controllably release the AA and the Al over time.

19. The method of claim 18, in which at least one of the AA or the Al is an mTor inhibitor.

20. The method of claim 18 in which the AA and the Al are present together within the same nano-particles.
